(19) United States
(12) Patent Application Publication
(10) Pub. No.: US 2016/0374388 A1
(43) Pub. Date: Dec. 29, 2016

(54) TERPENE PROFILE REPLICATION BLENDS AND THEIR APPLICATIONS

(71) Applicant: Andrew Jon Thomson, Garberville, CA (US)

(72) Inventor: Andrew Jon Thomson, Garberville, CA (US)

(21) Appl. No.: 15/173,600

(22) Filed: Jun. 3, 2016

Related U.S. Application Data
(60) Provisional application No. 62/170,393, filed on Jun. 3, 2015.
(60) Provisional application No. 62/170,380, filed on Jun. 3, 2015.

(51) Int. Cl. A24B 15/16; A61K 36/534; A61K 36/899; A61K 36/752; A24B 15/34; A61K 31/194; A61K 36/738; A61K 9/00; A24B 15/30; A24B 15/32; A61K 36/28; A61K 36/53

(52) U.S. Cl. CPC A24B 15/16; A61K 36/28; A61K 36/534; A61K 36/899; A61K 36/752; A61K 36/53; A61K 31/194; A61K 36/738; A61K 9/0095; A24B 15/303; A24B 15/32; A24B 15/34

(57) ABSTRACT

The embodiments herein relate to a method of preparing a terpene profile replication blend. The method comprises determining a combination of terpene for a predetermined application and blending a predetermined quantity of the terpene in a blender for a predetermined time. The combination of terpenes is selected from the group consisting of limonene, linalool, alpha humulene, alpha perpineole, pinene, bergatomene, a-bisbolol, borneol, camphene, d3-carene, a-caryophyllene, caryophyllene, citronellal, citronellol, elemene, eugenol, fenchol, myrcene, phellandrene, or terpinyl acetate.

### BACKGROUND

### Technical Field of Invention

The embodiments herein generally relate to terpenes and their properties and particularly to methods for developing terpene formulations. The embodiments herein more particularly relate to methods for terpene profiles replication and developing novel formulations for various applications.

### DESCRIPTION OF RELATED ART

An electronic cigarette or e-cigarette or e-cig is a handheld electronic device that vaporizes a flavoured liquid. The user inhales the vapour, which is called vaping. The fluid in the e-cigarette is called e-liquid or vapour fluid. E-liquid is usually made of nicotine, propylene glycol, glycerine, and flavourings.

The electronic cigarette industry, both nicotine based and any other effort to market similar technology with or without nicotine faces a problem. These products rely on an “inactive” base fluid in which the active ingredients are suspended in a shelf stable emulsion. In addition, these base fluids should possess the necessary chemical composition to function reliably in electronic or similar vaporization or atomization hardware.

Users by far prefer the sweet natural flavour for the base fluids. The Vegetable Glycerine or ‘VG’ is the most preferred base fluid because of its food based and nontoxic nature. Due to its likely low risk safety profile, being a common food product made from food, under maximally safe unregulated GRAS status under the FDA regulations, and also availability in a certified organic form allows vegetable glycerine to be used as a certified organic base e-cig fluid.

Unfortunately for manufacturers, VG is a botanical product, therefore highly inconsistent in chemical composition. The hydrophilic nature of the compound makes it easily and readily absorb the moisture even at ambient atmosphere. Thus VG does not function reliably in electronic or other vaporization or atomization technology such as “electronic cigarettes”. The varying and unpredictable moisture levels and other variables inherent in botanical products cause the fluids based on VG to have high variability in vaporization functional ability.

In addition, most desired active ingredient compounds used in electronic cigarettes and other vaporization technology include nicotinoids and other pharmacologically active compounds or further subjectively desirable compounds used in such products include flavourings, aromatics and preservatives, which are in most cases partly or entirely immiscible in the base of VG and thus does not remain in suspended form or in a shelf stable emulsion without the addition of toxic additives.

Thus the industry by far favours artificial and potentially toxic chemicals which are non-food alternatives to Vegetable Glycerine. These alternatives are limited in use due to their technological functional requirements. The industrial toxic base fluids are artificial, chemical or plastic in nature. The ones which are commercially reliable, are not hydrophilic in nature thus retain stable moisture levels and are generally miscible with the active ingredients, and these require no special methods to create shelf stable suspension and emulsion. However, by using such alternatives, the industry is causing great potential harm to consumers and liability risk to themselves as these alternatives are toxic, unsafe, unnatural and non-food base fluids. One such popular base fluid is Propylene Glycol or PG, also known as Antifreeze. A popular alternative certain manufacturers claim to be safer due to its use in certain pharmaceutical products is Polyethylene Glycol or PEG and similar related chemical compounds which are in layman's terms essentially liquefied plastics, fossil fuel derivatives, etc. The industrial chemicals with known toxicity including published peer reviewed medical evidence of increased cancer risk, and additional suspected health and safety dangers, as warned of in many recent statements by the FDA and other health and safety authorities. With all these negative factors and threats of increased regulation or outright bans, the industry and users desperately need an alternative base fluid.

Most of the users strongly prefer the naturally sugar free sweet flavour and likely health profile of food-based VG over the toxic chemical flavour and reported unhealthy side effects of the most common base fluids such as antifreeze PG and or plastic based PEG (including sore throat, lung pain, headaches, nausea, diarrhoea and increased cancer risk). Most users of electronic cigarette or other vaporization type products are at least partly motivated by harm reduction or health concerns, wanting to decrease health risks by “vaporizing” instead of smoking traditional cigarettes and in some cases seeking alternatives to toxic and addictive nicotine in these type of “e-cig” or “vape” or “e-juice” products. However, as the FDA and other health authorities warn, the chemicals used typically in these type of products may be dangerous, potentially even more dangerous than traditional cigarette smoking.

Therefore, there is a tremendous commercial need and public safety moral imperative to create commercially reliable and functional, food grade nontoxic or organic base fluids for e-cigarettes.

The above mentioned shortcomings, disadvantages and problems are addressed herein, as detailed below.

### SUMMARY OF THE INVENTION

The primary object of the embodiments herein is to provide a method for terpene replication and determining a formulation to various application using a plurality of terpenes.

Another object of the embodiments herein is to determine a predetermined formulation based on the various properties of the terpenes and replicating those properties to develop a novel formulation.

Yet another object of the embodiments herein is to develop formulations for various applications such as aromatherapy nasal inhalers, body care and cleaning products including soap, shampoo, air freshener, surface cleaner, night-time tea energizer, daytime tea energizer and decaffeinated daytime tea energizer.

Yet another object of the embodiments herein is to provide an organic vapour fluid composition for e-cigarettes comprising a plurality of terpenes.

Yet another object of the embodiments herein is to provide an organic vapour fluid composition for e-cigarettes that is non-toxic and natural.

Yet another object of the embodiments herein is to provide an organic vapour fluid composition for e-cigarettes with enhanced flavours and desired effects with reduced harms and unhealthy side effects.

Yet another object of the embodiments herein is to provide an organic vapour fluid composition for e-cigarettes that replaces nicotine and its harmful effects while smoking and is healthy for the users.

Yet another object of the embodiments herein is to provide an organic vapour fluid composition for e-cigarettes that is safer, more commercially reliable and industrially stable, avoids moisture and functionality problems in e-cigarette hardware.

Yet another object of the embodiments herein is to provide an organic vapour fluid composition for e-cigarettes which aids in the treatment of nicotine addiction.

According to an embodiment herein, a method of preparing a terpene profile replication blend comprises determining a combination of terpene for a predetermined application and blending a predetermined quantity of the terpene in a blender for a predetermined time. The combination of terpenes is selected from the group consisting of limonene, linalool, alpha humulene, alpha perpineole, pinene, bergatomene, a-bisbolol, borneol, camphene, d3-carene, a-caryophyllene, caryophyllene, citronellal, citronellol, elemene, eugenol, fenchol, myrcene, phellandrene, or terpinyl acetate.

The application includes aromatherapy nasal inhalers, body care and cleaning products including soap, shampoo, air freshener, surface cleaner, night-time tea energizer, daytime tea energizer and decaffeinated daytime tea energizer.

The application is an organic vapour fluid composition for e-cigarettes. The composition comprises terpenes and terpenoids. The terpenes and terpenoids are present in a quantity of at least 1% to 30% by weight.

According to an embodiment herein, a method of preparing an organic vapour fluid composition for e-cigarettes comprises blending a mixture of a plurality of added terpenes in a base fluid while applying ultrasonication. The plurality of terpenes are limonene, linalool, alpha humulene, alpha perpineole and pinene. The limonene is added in a quantity of 50% by weight. The linalool is added in a quantity of 20% by weight. The alpha humulene is added in a quantity of 10% by weight. The alpha terpineole is added in a quantity of 5% by weight. The pinene is added in a quantity of 15% by weight. The base fluid is vegetable glycerine.

According to another embodiment herein, a method for preparing terpene tea night time relaxer blend comprises blending a predetermined amount of chamomile, spearmint, lemongrass, orange peel, lavender and citric acid. The predetermined amount of chamomile is 55%. The predetermined amount of spearmint is 22.5%, The predetermined amount of lemongrass is 10.0%. The predetermined amount of orange peel is 5.0%. The predetermined amount of lavender is 2.5%. The predetermined amount of citric acid is 5.0%. The blending is done for at least 10 minutes.

According to another embodiment herein, a method for preparing daytime energizer terpene tea comprises blending a predetermined amount of green tea, peppermint, lemongrass, rosehips, lemon peel and citric acid. The predetermined amount of green tea is 50%. The predetermined amount of peppermint is 20.5%. The predetermined amount of lemongrass is 10.0%. The predetermined amount of rosehips is 10.0%. The predetermined amount of lemon peel is 5%. The predetermined amount of citric acid is 5.0%. The blending is done for at least 10 minutes.

According to another embodiment herein, a method for preparing a decaffeinated daytime energizer terpene tea comprises blending a predetermined amount of decaffeinated green tea, peppermint, lemongrass, rosehips, lemon peel and citric acid. The predetermined amount of green tea is 50%. The predetermined amount of peppermint is 20.5%. The predetermined amount of lemongrass is 10.0%. The predetermined amount of rosehips is 10.0%. The predetermined amount of lemon peel is 5%. The predetermined amount of citric acid is 5.0%. The blending is done for at least 10 minutes.

The embodiments herein provide an organic vapour fluid composition for e-cigarettes. The composition comprises a plurality of terpenes and a base fluid. The plurality of terpenes is limonene, linalool, alpha humulene, alpha perpineole and pinene. The base fluid is vegetable glycerine.

According to an embodiment herein, the limonene is present in a quantity of 50% by weight.

According to an embodiment herein, the linalool is present in a quantity of 20% by weight.

According to an embodiment herein, the alpha humulene is present in a quantity of 10% by weight.

According to an embodiment herein, the alpha terpineole is present in a quantity of 5% by weight.

According to an embodiment herein, the pinene is present in a quantity of 15% by weight.

According to an embodiment herein, the base fluid is vegetable glycerine.

According to an embodiment herein, the composition comprises terpenes and terpenoids. The terpenes and terpenoids are present in a quantity of at least 1% to 30% by weight.

According to another embodiment herein, a method of preparing an organic vapour fluid composition for e-cigarettes. The method comprises blending a mixture of a plurality of added terpenes in a base fluid while applying ultrasonication. The plurality of terpenes are limonene, linalool, alpha humulene, alpha perpineole and pinene. The base fluid is vegetable glycerine. The limonene is added in a quantity of 50% by weight. The linalool is added in a quantity of 20% by weight. The alpha humulene is added in a quantity of 10% by weight. The alpha terpineole is added in a quantity of 5% by weight. The pinene is added in a quantity of 15% by weight.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 is a graph showing the content of the terpenes used in replication a profile for a night-time tea relaxer formula, according to an embodiment herein.

FIG. 2 is graph showing the analysis of the Daytime energizer terpene tea formulation, according to an embodiment herein.

### DETAILED DESCRIPTION OF THE EMBODIMENTS

In the following detailed description, a reference is made to the accompanying drawings that form a part hereof, and in which the specific embodiments that may be practiced is shown by way of illustration. The embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The various embodiments herein provide a “Naturally Stabilized Food grade organic Vapour Fluid”, or a “vapour stabilized glycerine” or “terpene supplement for stabilizing and enhancing the vapour fluid” or “terpenized vapour fluid” or “E-cig fluid”. The embodiments herein provide a natural chemical formulation and a method of preparing a stabilized vapour fluid to be used in e-cigarettes. The embodiments herein provide an improved vapour fluid composition having a stable shelf life and functionality which can be used in all kinds of e-cigarettes hardware.

The embodiments herein provide an organic vapour fluid composition for e-cigarettes. The composition comprises a plurality of terpenes and a base fluid. The plurality of terpenes is limonene, linalool, alpha humulene, alpha perpineole and pinene. The base fluid is vegetable glycerine. The limonene is present in a quantity of 50% by weight. The linalool is present in a quantity of 20% by weight. The alpha humulene is present in a quantity of 10% by weight. The alpha terpineole is present in a quantity of 5% by weight. The pinene is present in a quantity of 15% by weight. The base fluid is vegetable glycerine.

According to an embodiment herein, the composition comprises terpenes and terpenoids. The terpenes and terpenoids are present in a quantity of at least 1% to 30% by weight.

According to an embodiment herein, the method of preparing an organic vapour fluid composition for e-cigarettes comprises blending a mixture of a plurality of added terpenes in a base fluid while applying ultrasonication. The plurality of terpenes are limonene, linalool, alpha humulene, alpha perpineole and pinene. The base fluid is vegetable glycerine. The limonene is added in a quantity of 50% by weight. The linalool is added in a quantity of 20% by weight. The alpha humulene is added in a quantity of 10% by weight. The alpha terpineole is added in a quantity of 5% by weight. The pinene is added in a quantity of 15% by weight.

According to an embodiment herein, natural terpenes are used as the stabilizing ingredients for the preparation of a vapour fluid for e-cigarettes.

In the present invention, by conducting a scientific testing or “quantitative” analysis of all aromatic plants and all herbal ingredients or aromatherapy essential oils, a relative percentage of each terpene present in the original plant or essential oil is established. Then, by using these percentages found in nature, same ratios of these terpenes are recreated by mathematical analysis and artificially blending the individual terpenes to recreate the natural matrix formulated by natural plant evolution.

“Terpene Matrix Replication”, “Terpene Profile imitation” or “Natural Botanical Effect Replication” or “Natural Terpene Profile Replication” is the concept of blending natural chemical formulations based on partly or totally, approximately or exactly replicating the active natural chemical matrix or “terpene profile” of a botanical herb, essential oil or aromatherapy product. This is done by measuring the relative percentages of certain key terpenoid compounds in those aromatic plants and then copying those percentages by manually blending purified versions of the same compounds from the same or other sources. This is done to replicate or simulate the evidence based scientifically verified pharmacological and subjective psychological effects of the targeted original botanical product or herbal/aromatherapy product. These herbal products can be combined partially in combination with partial or total replications of other pharmacologically or psychologically useful botanical terpene profiles imitating natural ones created through plant evolution to create consumer friendly, effect based, or botanical plant type based products which the gives the users the desired effects similar to those of the replicated botanicals. So that users and manufacturers of the natural nutritional and aroma therapeutic products, or to produce similar health and wellness qualities or benefits through incidental exposure and absorption through skin and breathing air when used in body care or cleaning products in the home or business environment. or added to e-cig vapour fluid, nasal inhaler products, infusion into foods or beverages such as infused tea, “smart drinks” or “energy drinks” and other direct supplementation methods. The terpene replication has many applications from aromatherapy nasal inhalers using a vented plastic tube with cotton insert to store a terpene profile replication blend for convenient user inhalation, to infusion into body care and cleaning products like soap, shampoo, air freshener, surface cleaner etc. for incidental absorption to replicate botanical effects and aesthetic scent or flavor qualities.

Another popular application is putting it into a beverage, dietary supplement, food or other consumable product to replicate reliably the approximate effects of consuming a targeted “whole plant” natural botanical extract but without the drawbacks.

In the present specification, the word “terpenes” to refer to monoterpenes, diterpenes, terpenoids etc, any type of molecule with repeating isoprene units also known as terpenes of any kind are referred to as “terpenes”.

Terpenes are natural products derived from plants that have medicinal properties and biological activity. Terpenes may be found in cleaning products, rubefacients, aromatherapy, and various topical preparations. Terpenes may exist as hydrocarbons or have oxygen-containing compounds such as ketone or aldehyde groups (terpenoids). The basic structure of terpenes is repeating isoprene units (C5H8)n, and they are grouped according to the number of repeating isoprene units. Monoterpenes contain 2 isoprene units; examples include cantharidin, menthol, pinene, and camphor. Diterpenes contain 4 isoprene units; examples include phytol, vitamin A, and paclitaxel (Taxol).

The same shelf stable emulsification method of ultrasonic vibration or “ultrasonication” here again allows permanent emulsification and active ingredient suspension without the use of added emulsifiers or toxic base fluids which would compromise the healthy, natural ad nontoxic qualities attainable with the present invention.

Terpenes are the largest and the most diverse group of chemical compounds found in nature. Much of the history and practice of the herbal and nutritional medicine, as well as the evidence based, scientific approaches to so called aromatherapy, derives much of it's scientifically identified pharmacological and subjective psychological benefits from the terpenes and terpenoid compounds found within all natural aromatic plants, especially those used as supplements and medicinal or therapeutic applications.

The ultrasonication temperatures must be above 30 Fahrenheit to blend properly and under 100 Fahrenheit to preserve terpenes, ideally under 75 degrees Fahrenheit and over 45 degrees Fahrenheit. The duration of ultrasonication varies depending on specific blend, and brand/specs of the ultrasonication machine being used. The maximum practical vibration settings are used a minimum of 30 seconds to a maximum of 30 minutes is typical but depends entirely on volume of container vessel, degree of immiscibility and volatility/solvent action of any particular blend of fluids and the mass being ultrasonicated, and is considered complete and successful when the compounds no longer separate when undisturbed for 48 hours.

To be effective, the total terpene or terpenoid content, depending on which terpenes are chosen for a particular flavour or effect, must be sufficient to increase the volatility of the Vegetable Glycerine (VG) or the base fluid sufficiently and reduce the hydrophilic effect sufficiently to achieve the desired results. These ratios inherently vary due to variability of the VG being treated. The varying volatility and flavours of various similarly usable terpene blends and other similar variables is between 1% to 30% of these terpenes or terpenoids. This range of terpenes or terpenoids used gives the desired results.

In addition, this wide variety of terpene blends can be used without nicotine or other active ingredients providing the user with desirable pharmacological or subjective psychological benefits. As demonstrated by the vast body of medical scientific literature on terpenes used for a century in the food and perfume industries and the various herbal or aromatherapy effects. The terpene is found to have properties to curb nicotine cravings when used in a combination with sedatives. The terpenes linalool and alpha humulene used in the present invention have sedative effects while the terpenes pinene, menthene and alpha terpineole have simulating effects. These terpenes help in blocking the ability of the user to consume nicotine and biologically blocking the nicotine urge for tobacco cessation. In addition, these terpenes based blends are used for a myriad of other scientifically proven health benefits or harm reductions. The use of the Terpene D-Limonene is proven scientifically to be a powerful anti-carcinogen and natural anti-depressant while providing extremely effective volatilization, stabilization and increased palatable flavour qualities.

The immiscibility of fluids is overcome by a combination of the highly solvent nature of the terpenes and terpenoids with a physical process known as ultrasonic vibration or ultrasonication to achieve shelf stable emulsification and active ingredient suspension without the use of any additives or non-food type base fluid. The “ultrasonication” is used to create shelf-stable emulsification without toxic chemicals or emulsifiers of any kind, with the additional benefit that these additives have scientifically proven health and wellness benefits for the consumer to reduce harm or even replace and quit tobacco or nicotine products entirely, such as blends using compounds from the botanical ingredient “Lobelia”.

Some terpenes have some toxicity despite “safe” FDA status. Thus care must be taken in selecting terpenes for specific applications. Camphor in particular should be avoided at all costs and not used in the present innovative formulation method because of known toxicity and large numbers of adverse incidents resorted to poison control authorities involving camphor and products containing it such as whole pine oil.

Generally, d-limonene is the safest, most affordable and abundant, most effective and widely applicable terpene for most applications. The safety/health profile is safest and most beneficial, according to the publicly available evidence, the flavor, particularly in ultra high purity versions is the most neutral and universally palatable, and is widely compatible with various terpene replication blends without overpowering their flavor profiles or used by itself to achieve vapor fluid stabilization and functional improvement without other terpenes if appropriate.

Pinene is a good alternative or additive to Limonene due to shared stimulating effects and relatively palatable neutral flavor.

However, limonene and myrcene are stimulating, so not ideal for sedating its relaxing blends, for which the best alternatives are humulene (alpha and beta) as well as myrcene which are relaxing or sedating in effect and have relatively mild neutral or at least highly palatable flavor profiles.

The top safest, most appropriate and ideal, most available and abundant, most scientifically investigated, top most useful and palatable terpenes for this and their basic laypersons science details are below:

Alpha-Pinene, Beta-Pinene: The aroma is pine, and has alertness, memory retention, counteracts some THC effects. The terpene has medicinal properties in asthma, and antiseptic. The terpene is also found in pine needles, rosemary, basil, parsley, dill.

Myrcene: has musky, cloves, earthy, herbal with notes of citrus and tropical fruit aroma. It is sedating “couchlock” effect, and relaxing effect. The medicinal value is antioxidant, anti-carcinogenic; good for muscle tension, sleeplessness, pain, inflammation, and depression. The terpene is found in mango, lemongrass, thyme, and hops.

Limonene: The aroma is citrus and has elevated mood, and stress relief effects.

Caryophyllene: The aroma is pepper, spicy, woody, cloves.

Linalool: The aroma is floral, citrus, and candy. It has anxiety relief and sedative effects. The linalool is best for aroma based, topical and environmental applications of terpene profile replication (aka terpene-power blends) not ideal for internal consumption.

β-Caryophyllene: Beta-caryophyllene is a sesquiterpene found in many plants including Thai basils, cloves and black pepper, and has a rich spicy odor. Research has shown that β-Caryophyllene has affinity for the CB2 endocannabinoid receptor. β-Caryophyllene is known to be anti-septic, anti-bacterial, antifungal, anti-tumor and anti-inflammatory.

β-Myrcene: β-Myrcene is a monoterpene, and for a wide variety of reasons, one of the most important terpenes. It is a precursor in the formation of other terpenes, as well. β-Myrcene is found fresh mango fruit, hops, bay leaves, eucalyptus, lemongrass and many other plants. β-Myrcene is known to be anti-tumor, anti-inflammatory, and used in the treatment of spasms. It is also used to treat insomnia, and pain. It also has some very special properties, including lowering the resistance across the blood to brain barrier, allowing itself and many other chemicals to cross the barrier easier and more quickly. β-Myrcene can be used in this same manner to improve uptake with a wide variety of chemical compounds.

D-Limonene: D-limonene is a cyclic terpene of major importance with a strong citrus odor and bitter taste. D-limonene was primarily used in medicine, food and perfume until a couple of decades ago, when it became better known as the main active ingredient in citrus cleaner. It has very low toxicity, and humans are rarely ever allergic to it. Medicinally, Limonene is best known for treating gastric reflux and as an anti-fungal agent. It's ability to permeate proteins makes it ideal for treating toenail fungus. Limonene is also useful in treating depression and anxiety. Limonene also assists in the absorption of other terpenoids and chemicals through the skin, mucous membranes and digestive tract. It's also been shown to be effective anti-tumor while at the same time being an immunostimulant. Limonene is one of two major compounds formed from α-Pinene.

Humulene: Humulene is a sesquiterpene also known as α-humulene and α-caryophyllene; an isomer of β-caryophyllene. Humulene is found in hops and Vietnamese coriander, among others. Humulene gives beer its ‘hoppy’ aroma. It is anti-tumor, anti-bacterial, anti-inflammatory, and anorectic (suppresses appetite). It has commonly been blended with β-caryophyllene and used as a major remedy for inflammation, and is well known to Chinese medicine.

α-Pinene: α-Pinene is one of the principle monoterpenes, and is important physiologically in both plants and animals, and to our environment. α-Pinene tends to react with other chemicals, forming a variety of other terpenes (like D-Limonene) and other compounds. α-Pinene has been used for centuries as a bronchodilator in the treatment of asthma. α-Pinene is also anti-inflammatory. It is found in conifer trees, orange peels among others, and known for it's sharp sweet odor. α-Pinene is a major constituent in turpentine.

Pulegone: Aroma is mint, camphor, rosemary, candy. It is implicated in liver damage in very high dosages. Pulegone is an acetylcholinesterase inhibitor. That is, it stops the action of the protein that destroys acetylcholine, which is used by the brain to store memories.

Sabinene—Found in oak trees, tea tree oil, black pepper and is a major constituent of carrot seed oil.

Terpineol: The aroma is floral, lilac, citrus, apple/orange blossoms, and lime. It is a minor constituent of many plant essential oils. It is used in perfumes and soaps for fragrance. It reduces physical motility 45% in lab rat tests.

Borneol: The aroma is menthol, camphor, pine, and woody. Can be easily converted into menthol. Borneol is found in Cinnamon and Wormwood. It is considered a “calming sedative” in Chinese medicine. It is directed for fatigue, recovery from illness and stress.

Cineole/Eucalyptol: Spicy, camphor, refreshing, minty. Found in rosemary, eucalyptus. It is used to increase circulation, pain relief and easily crosses the blood-brain-barrier to trigger fast olfactory reaction. Eucalyptus oil is considered centering, balancing and stimulating.

Delta-3-Carene—sweet, pine, cedar, woodsy, pungent. A constituent of rosemary, pine and cedar resin. In aroma therapy, cypress oil, high in D-3-carene, is used to dry excess fluids, tears, running noses, excess menstrual flow and perspiration.

Science has discovered hundreds of terpenes, for profile replication the most practical ones (based on safety profiles, cost/abundance/availability and on consumer desirability as measured by their biomedical and psychological effects, their palatability/flavor/scent and on general consumer awareness/popularity.

The present invention provides concept and method results in different formulations for each and every application. For example, according to an embodiment herein, a terpene profile replication blend to infuse into bottled tea beverages is provided. According to another embodiment herein, terpene vapor stabilization with five existing flavor blends are provided in which terpene replication blends can be infused.

Sesquiterpenes and hemiterpenes are also used in the present invention.

In the present invention, quantitative headspace analysis is performed to determine the 2-12 most prevalent terpenes in their existing flavors and then blend custom terpene blends using those exact same ratios found in the analysis test data.

The best embodiment depends on specific application. For terpene profile replication, a sedating blend imitating lavendar would be dominant in linalool but also contain several other terpenes. A stimulating blend imitating mint would be highest in menthene but also contain several other terpenes.

For e-cig/vapor stabilization any GRAS food grade terpene works, limonene has a relatively neutral flavor so is more widely applicable, but there are dozens that is in use, depending on the application. For example, in a lemon/lime beverage, limonene dominant. The popular sedative terpene linalool is useful in terpene replication blends not designed for oral consumption, but not in dominant ratio in vapor/e-cig products or other flavor sensitive applications due to unpalatability.

But in practice, any embodiment would be formulated by simply using a terpene profile test from a real botanical and approximating it by recombining the main terpenes in it from individual purified sources which means that the formulation method is simple and consistent but each application required individual customized formulating.

The detailed real world examples of natural terpene profile approximations with specific numerical ratios and graphs are provided below. But the applications that exist are as varied as the world of botanical plants themselves.

### Example 1

An analysis of a normal herbal tea product “terpene tea night time relaxer formula” with normal ingredients was done. The percentages of terpenes measured is as follows, and would be used to determine exact (same) percentages in the replication blend.

Table 1 shows the formulation replicated for night-time tea relaxer.

TABLE 1
Formula for night-time tea relaxer

| Ingredients | Percentage | Total Pounds |
|---|---|---|
| Organic Chamomile | 55.0% | 55 |
| Organic Spearmint | 22.5% | 23 |
| Organic Lemongrass | 10.0% | 10 |
| Organic orangepeel | 5.0% | 5 |
| Lavender | 2.5% | 3 |
| Citric Acid | 5.0% | 5 |

The ribbon blender was used for blending. The blending was done for 10 minutes.

Table 2 shows the natural whole form ingredients measured in a tea blend in the following order of proportion, largest to smallest:

TABLE 2
Terpene tea night time relaxer formula

| | |
|---|---|
| Bergatomene | 0.00 |
| a-Bisbolol | 12.37 |
| Borneol | 0.00 |
| Camphene | 0.00 |
| D3-Carene | 0.00 |
| a-caryophyllene | 0.00 |
| Caryophyllene | 0.00 |
| Citronellal | 0.00 |
| Citronellol | 0.00 |
| Elemene | 0.67 |
| Eugenol | 0.00 |
| Fenchol | 0.00 |
| Limonene | 12.67 |
| Linalool | 8.63 |
| Myrcene | 0.00 |
| Phellandrene | 0.00 |
| Pinene | 1.27 |
| Terpinyl Acetate | 0.00 |

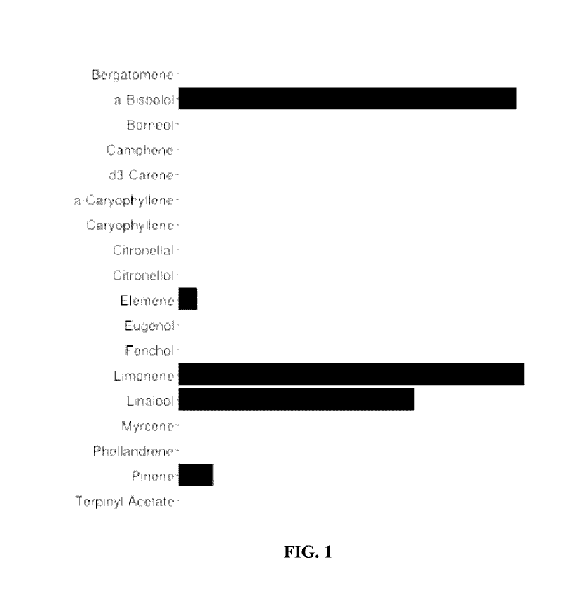

FIG. 1 is a graph showing the content of the terpenes used in replication a profile for a night-time tea relaxer formula, according to an embodiment herein. With respect to FIG. 1, the terpenes were tested for total terpene content ratios and the numbers in the test result need merely to be duplicated but with the terpenes as purified samples or bulk pure terpenes to create a terpene power replication of this profile which will similarly relax the user without access to the tea, enhance the effects of the tea on users, or alter or reverse the effects of a stimulating tea.

### Example 2

A “daytime energizer terpene tea” formula was replicated. The ingredients were green tea, peppermint, lemongrass, rose hips, and lemon peel.

Table 3 shows the formulation replicated for daytime energizer terpene tea:

TABLE 3
Formula for daytime energizer terpene tea

| Ingredients | Percentage | Total Pounds |
|---|---|---|
| Organic Green Tea | 50.0% | 50 |
| Organic Peppermint | 20.0% | 20 |
| Organic Lemongrass | 10.0% | 10 |
| Rosehips | 10.0% | 10 |
| Organic lemon peel | 5.0% | 5 |
| Citric Acid | 5.0% | 5 |

The ribbon blender was used for blending. The blending was done for 10 minutes.

In case of de-caffeinated daytime terpene tea blend, a decaf. Green tea is used in the same percentage.

Table 4 shows the ingredients and their percentages:

TABLE 4
Daytime energizer terpene tea

| | |
|---|---|
| Bergatomene | 0.80 |
| a-Bisbolol | 0.00 |
| Borneol | 0.09 |
| Camphene | 19.64 |
| D3-Carene | 0.00 |
| a-caryophyllene | 0.00 |
| Caryophyllene | 0.00 |
| Citronellal | 0.56 |
| Citronellol | 1.55 |
| Elemene | 0.00 |
| Eugenol | 0.00 |
| Fenchol | 0.00 |
| Limonene | 1.10 |
| Linalool | 0.00 |
| Myrcene | 0.00 |
| Phellandrene | 0.28 |
| Pinene | 2.44 |
| Terpinyl Acetate | 0.00 |

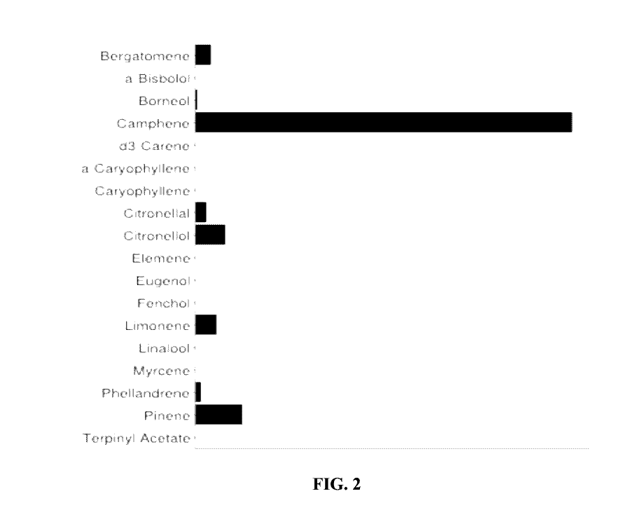

The analysis of the terpene content was done. FIG. 2 is graph showing the analysis of the Daytime energizer terpene tea formulation, according to an embodiment herein. With respect to FIG. 2, it was found that the formulation was having a high levels of camphene. So as stated previously, camphene toxicity and high numbers of adverse events reported, hence this formulation was made an exception and camphene was not suggested to be included in the formulation used for day time energizer terpene tea. Instead, menthene was substituted at the same percentage.

It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the claims.

I claim:

1. A method of preparing a terpene profile replication blend, comprises:
determining a combination of terpene for a predetermined application;
blending a predetermined quantity of the terpene in a blender for a predetermined time.

2. The method as claimed in claim 1, wherein the combination of terpenes is selected from the group consisting of limonene, linalool, alpha humulene, alpha perpineole, pinene, bergatomene, a-bisbolol, borneol, camphene, d3-carene, a-caryophyllene, caryophyllene, citronellal, citronellol, elemene, eugenol, fenchol, myrcene, phellandrene, or terpinyl acetate.

3. The method as claimed in claim 1, wherein the application includes aromatherapy nasal inhalers, body care and cleaning products including soap, shampoo, air freshener, surface cleaner, night-time tea energizer, daytime tea energizer and decaffeinated daytime tea energizer.

4. The method as claimed in claim 1, wherein the application is an organic vapour fluid composition for e-cigarettes, composition according to claim 1, wherein the composition comprises terpenes and terpenoids, wherein the terpenes and terpenoids are present in a quantity of at least 1% to 30% by weight.

5. A method of preparing an organic vapour fluid composition for e-cigarettes, comprises:
blending a mixture of a plurality of added terpenes in a base fluid while applying ultrasonication, wherein the plurality of terpenes are limonene, linalool, alpha humulene, alpha perpineole and pinene.

6. The method according to claim 5, wherein the limonene is added in a quantity of 50% by weight.

7. The method according to claim 5, wherein the linalool is added in a quantity of 20% by weight.

8. The method according to claim 5, wherein the alpha humulene is added in a quantity of 10% by weight.

9. The method according to claim 5, wherein the alpha terpineole is added in a quantity of 5% by weight.

10. The method according to claim 5, wherein the pinene is added in a quantity of 15% by weight.

11. The method according to claim 5, wherein the base fluid is vegetable glycerine.

12. The method according to claim 5, wherein the method further comprises a method for preparing terpene tea night time relaxer blend, wherein the method for preparing terpene tea night time relaxer blend comprises:
blending a predetermined amount of chamomile, spearmint, lemongrass, orange peel, lavender and citric acid,
wherein the predetermined amount of chamomile is 55%,
wherein the predetermined amount of spearmint is 22.5%,
wherein the predetermined amount of lemongrass is 10.0%,
wherein the predetermined amount of orange peel is 5.0%,
wherein the predetermined amount of lavender is 2.5%,
wherein the predetermined amount of citric acid is 5.0%;
and wherein the blending is done for at least 10 minutes.

13. The method according to claim 5, wherein the method further comprises a method for preparing daytime energizer terpene tea, wherein the method for preparing daytime energizer terpene tea comprises:
blending a predetermined amount of green tea, peppermint, lemongrass, rosehips, lemon peel and citric acid,
wherein the predetermined amount of green tea is 50%,
wherein the predetermined amount of peppermint is 20.5%,
wherein the predetermined amount of lemongrass is 10.0%,
wherein the predetermined amount of rosehips is 10.0%,
wherein the predetermined amount of lemon peel is 5%,
wherein the predetermined amount of citric acid is 5.0%;
and wherein the blending is done for at least 10 minutes.

14. A method for preparing a decaffeinated daytime energizer terpene tea, comprises:
blending a predetermined amount of decaffeinated green tea, peppermint, lemongrass, rosehips, lemon peel and citric acid,
wherein the predetermined amount of green tea is 50%,
wherein the predetermined amount of peppermint is 20.5%,
wherein the predetermined amount of lemongrass is 10.0%,
wherein the predetermined amount of rosehips is 10.0%,
wherein the predetermined amount of lemon peel is 5%,
wherein the predetermined amount of citric acid is 5.0%;
and wherein the blending is done for at least 10 minutes.
